(19) United States
(12) Patent Application Publication
(10) Pub. No.: US 2024/0091039 A1
(43) Pub. Date: Mar. 21, 2024

(54) DEVICES IMPLANTABLE ACROSS DYNAMIC ANATOMICAL PASSAGES AND ASSOCIATED SYSTEMS AND METHODS

(71) Applicant: BOSTON SCIENTIFIC SCIMED, INC., MAPLE GROVE, MN (US)

(72) Inventors: Yeison Calvo, San Ramón (CR); John Thomas Favreau, Shrewsbury, MA (US)

(73) Assignee: BOSTON SCIENTIFIC SCIMED, INC., MAPLE GROVE, MN (US)

(21) Appl. No.: 18/369,480

(22) Filed: Sep. 18, 2023

Related U.S. Application Data
(60) Provisional application No. 63/407,948, filed on Sep. 19, 2022.

(51) Int. Cl. A61F 5/00

(52) U.S. Cl. CPC A61F 5/0079; A61F 5/0036

(57) ABSTRACT

An implantable medical device having at least one compliant retention member configured not only to resist migration of the device with respect to its deployment site, but also to conform to anatomical forces applied thereto. The retention member may present a convex tissue-contacting surface to tissue at the deployment site and to which anatomical forces may be applied. The retention member may be compressible upon application of radially-inwardly directed anatomical forces thereto, but is sized, shaped, configured, and/or dimensioned to withstand forces tending to cause migration of the implantable medical device even when being compressed radially-inwardly. As such, the implantable medical device withstands forces applied thereto without building up internal stresses which may result in material fatigue.

### CROSS-REFERENCE TO RELATED APPLICATIONS

This application claims the benefit of priority under 35 U.S.C. § 119 to U.S. Provisional Application No. 63/407,948, filed Sep. 19, 2022, the entire disclosure of which is hereby incorporated by reference herein for all purposes.

### FIELD

The present disclosure relates generally to the field of implantable medical devices. More particularly, the present disclosure relates to medical devices implantable across or within a dynamic anatomical passage capable of moving and exerting forces on the implantable medical device, and associated systems and methods.

### BACKGROUND

Various medical procedures or treatments involve placing an implantable medical device with respect to (across, through, etc.) an anatomical passage, such as to occlude flow of materials through such passage. For instance, treatment methods for various medical conditions, such as obesity, diabetes, or duodenal ulcers, involve restricting flow of materials through the duodenum or even bypassing the duodenum. In the case of a gastric bypass, a duodenal occlusion or exclusion device may be placed in the pyloric sphincter to inhibit or block passage of materials (fluid, chyme, etc.) from the stomach through the pylorus and into the duodenum. Gastric materials are redirected to a bypass, such as a gastrojejunal bypass formed by an anastomosis between the stomach and a portion of the jejunum. Various challenges to preventing migration of a deployed occlusion device are presented by the natural movements of the body (e.g., the gastrointestinal system) as well as the constant flow of materials against the occlusion device. Peristaltic movement of the pylorus to pass materials therethrough (e.g., distally into the small intestine), generally less frequent reverse peristalsis through the pylorus (proximally into the stomach), as well as the natural tendency of the pylorus to eject materials therein, present particular challenges for placement and retention of pyloric occlusion devices. However, formation of an implantable device to be resistant to migration may impact the ability of the implantable device to withstand repetitive movements or waves of anatomical forces impacting the device. For instance, a medical device implanted with respect to a pylorus typically must withstand regular/continuous peristaltic waves from the antrum of the stomach. Such forces may particularly impact welds of the implantable medical device and/or relatively stiff sections and/or angled surfaces of the implantable medical device designed to resist forces and thereby to retain the device in place against forces tending to cause migration of the device. Constant, continuous, and/or regular impact of forces on the implantable medical device may cause compression or deformation of regions of the device which, if excessive, may cause fatigue and/or failures of the device. Accordingly, there is an ongoing need for solutions to the various challenges faced by medical devices implanted with respect to anatomical passages, particularly dynamic anatomical passages which move periodically or frequently.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an implantable medical device includes a body having a first end, a second end, and a midsection therebetween. A first retention member extends from the first end of the body; and a second retention member extends from the second end of the body. In some aspects, the first retention member extends radially outwardly from the first end of the body and toward the midsection of the body to define a tissue-contacting surface with a surface area and flexibility sufficient to absorb radially-inwardly directed anatomical forces applied thereto; and the first retention member is configured to resist being inverted into a configuration with the free end thereof extending away from the first end and the midsection of the body.

In some embodiments, the first retention member is formed of a compliant material capable of flexing with radially-inwardly directed anatomical forces applied thereto. In some embodiments, the first retention member is compressible from an initial expanded configuration in response to application of radially-inwardly forces applied thereto, and is formed of a resilient material returning the first retention member to the expanded configuration upon the force subsiding. In some embodiments, the first retention member presents a convex tissue-contacting surface to tissue at the deployment site, and is configured and dimensioned to resist migration from the deployment site.

In some embodiments, the body is formed of a tubular member, and at least the first retention member is formed by inverting an end of the tubular member and extending the free end of the inverted end toward the midsection of the tubular member a sufficient distance to cause the inverted end of the tubular member to resist returning to its initial uninverted tubular configuration. In some embodiments, the tubular member is formed from a plurality of interwoven filaments.

In some embodiments, the first retention member is formed from a bowl-shaped element separate from the body. In some embodiments, the first retention member is movable with respect to the body. In some embodiments, the body is formed from an elastic material allowing the first retention member to be moved away from the second retention member.

In some embodiments, the tissue-contacting surface of the first retention member is convex.

In accordance with various principles of the present disclosure, an implantable medical device, configured to be implanted with respect to a pylorus of a patient, includes a body having a first end, a second end, and a midsection extending therebetween; a gastric retention member extending radially-outwardly from the first end of the body; and a duodenal retention member extending radially-outwardly from the second end of the body. In some embodiments, the gastric retention member has a tissue-contacting surface extending radially outwardly from the first end of the body and toward the midsection of the body to a free edge of the gastric retention member spaced from the first end of the body in a direction toward the midsection of the body a sufficient extent to remain in such configuration when the implantable medical device is implanted with respect to the patient's pylorus with the gastric retention member positioned within the patient's stomach. In some embodiments, the gastric retention member is resiliently compliant to contract from an expanded configuration to a compressed configuration upon application of peristaltic forces thereto from the antrum of the patient's stomach, and to return to the expanded configuration upon passing of a peristaltic wave from the gastric retention member.

In some embodiments, the gastric retention member presents a convex tissue-contacting surface to the antrum of the patient's stomach.

In some embodiments, the implantable medical device is formed from a tubular member having a first end, a second end, and a midsection therebetween; and the gastric retention member is formed by inverting the first end of the tubular member and pulling the free end thereof towards the second end of the tubular member a sufficient distance to remain in the inverted configuration when implanted with respect to the antrum of the patient's stomach and when compressed by a peristaltic wave of the antrum.

In some embodiments, the body and at least the gastric retention member are formed separately and coupled together to allow relative movement therebetween. In some embodiments, the body is formed of an elastic material allowing the gastric retention member to move away from the duodenal retention member.

In accordance with various principles of the present disclosure, a method of at least partially occluding flow of materials through a pylorus of a patient includes deploying a body of an implantable medical device across the pylorus; deploying a gastric retention member of the implantable medical device in the antrum of the patient; and deploying a duodenal retention member of the implantable medical device in the duodenum of the patient. In some aspects, the gastric retention member has a tissue-contacting surface extending radially-outwardly from a first end of the body of the implantable medical device and towards a midsection of the body, and the gastric retention member is formed of a compliant material compressing in response to peristaltic contractions of the antrum and returning to an expanded configuration upon passing of a peristaltic contraction of the antrum. In some aspects, the duodenal retention member extends radially-outwardly from a second end of the body of the implantable medical device.

In some aspects, the implantable medical device is formed from a tubular member having a first end and a second end, with the first end inverted to form the gastric retention member of the implantable medical device, the method comprising placing the inverted first end in contact with the antrum with the free end of the tubular member positioned inwardly of the first end of the tubular member in a direction toward the second end of the tubular member.

In some aspects, the gastric retention member presents a convexly curved tissue-contacting surface, the method comprising placing the concavely-curved tissue-contacting surface in contact with the patient's antrum for contracting in response to peristaltic waves applied thereto.

In some aspects, the gastric retention member extends a sufficient distance from the first end of the body to the midsection of the body to resist inversion causing the gastric retention member to extend away from the midsection of the body.

In some aspects, the implantable medical device is formed from a body separate from the gastric retention member such that upon placement of the implantable medical device with respect to the patient's pylorus, the gastric retention member is capable of movement with respect to the body of the implantable medical device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery device such as a catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of an example of an embodiment of an implantable medical device formed in accordance with various aspects of the present disclosure and positioned in a schematic representation of a gastrointestinal environment.

FIG. 2 illustrates an elevational view of an example of an embodiment of an implantable medical device such as illustrated in FIG. 1 with biological forces schematically illustrated as being applied thereto.

FIG. 3 illustrates a cross-sectional view along line III-III of the example of an embodiment of an implantable medical device illustrated in FIG. 2.

FIG. 4 illustrates an example of an embodiment of an implantable medical device formed in accordance with various principles of the present disclosure.

FIG. 5 illustrates an example of an embodiment of an implantable medical device formed in accordance with various principles of the present disclosure.

FIG. 6 illustrates a cross-sectional view along line VI-VI of the example of an embodiment of an implantable medical device illustrated in FIG. 5.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, including regions in the vicinity thereof, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along or within may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

The present disclosure relates to implantable medical devices having a body with a first end, a second end, and a midsection therebetween. The implantable medical device may be configured to be expandable from a collapsed or contracted delivery configuration (e.g., facilitating intraluminal/transluminal delivery to a deployment site through body passages without requiring open surgery for delivery of the device) to an expanded deployed configuration. The midsection of the implantable medical device is deployable across or along or at (such terms being used interchangeably herein without intent to limit) a deployment site within a body with a retention member at one or both ends thereof extending radially outward therefrom to retain the implantable medical device in place at the deployment site. It will be appreciated that reference may be made herein to a deployment site, anatomical site, delivery site, implant/implantation site, site of implantation, target site, treatment site, etc., interchangeably and without intent to limit. A non-limiting example of a deployment site referenced herein is an anatomical passage, which may be any passage or lumen within a patient's body, and may be referenced alternatively herein as a body passage, body lumen, anatomical lumen, etc., without intent to limit. It will be appreciated that reference to a body passage includes naturally-existing passages (e.g., the pylorus) as well as medically-created passages (e.g., a passage created with the use of a medical instrument, such as between a stomach and jejunum) or otherwise. One or both ends of the implantable medical device have a retention member configured to engage tissue walls surrounding ends of the anatomical passage to hold the implantable medical device in place and to resist migration of the implantable medical device from its deployment site.

At least a portion of various anatomical deployment sites in which an implantable medical device may be deployed may be subjected to various forces, movements, etc., which may put pressure or stress on the implantable medical device. Over extended periods of time, an implantable medical device subjected to forces at its deployment site (e.g., from dynamic anatomical tissue at the deployment site) may exhibit degradation, fatigue, or even points of failure. For instance, some implantable medical devices are formed of woven filaments welded at the free ends of the device to finish and hold the woven configuration in place without unravelling. Repeated and prolonged pressure on such devices may ultimately affect the welds at the free ends, potentially causing at least partial unravelling of the woven device.

In accordance with various principles of the present disclosure, at least one retention member of an implantable medical device is compliant and configured to flex in response to a force applied thereto, rather than to resist the force, while also still holding the implantable medical device in place at the deployment site. It will be appreciated that the term flex (including other grammatical forms thereof) is used herein for the sake of convenience and broadly and generally covers a level of compliance allowing the device to move with or to “ride” with and/or absorb forces in contrast with resisting forces, various such terms thus being used interchangeably herein without intent to limit. Without being bound by theory, flexing in response to forces such as naturally-occurring anatomical forces (e.g., peristaltic waves in the case of placement in the gastrointestinal tract), rather than resisting forces, may lead to less stress and thus less wear and tear and potential degradation.

More particularly, various anatomical deployment sites may subject a device implanted at such site to forces such as radially-inwardly directed forces. In accordance with various principles of the present disclosure, at least one retention member of an implantable medical device is sufficiently compliant to move with or to “ride” with anatomical forces applied thereto and to be compressed by such forces, rather than resist such forces. The at least one retention member is sized, shaped, configured, and/or dimensioned to resist migration from the deployment site even if compressed.

For the sake of simplicity, some descriptions herein are with reference to “a” retention member rather than to both retention members. It should be appreciated that such references are not intended to be limited to a single retention member, and may apply to both retention members of an implantable medical device formed in accordance with various principles of the present disclosure.

In some aspects, at least one retention member of an implantable medical device formed in accordance with various principles of the present disclosure presents an extended tissue-contacting surface for contacting tissue at the deployment site of the device. For instance, in some embodiments, a midsection of an implantable medical device is deployed across an anatomical passage, with a retention member expanded outside the anatomical passage and in contact with tissue surrounding an opening into the anatomical passage. In accordance with various principles of the present disclosure, the tissue-contacting surface of the retention member extends from a generally central region thereof (optionally adjacent the longitudinal axis of the implantable medical device and/or the body thereof) radially-outwardly from the longitudinal axis and longitudinally away from the end of the implantable medical device from which the retention member extends and towards the midsection of the implantable medical device and the opposite end of the implantable medical device. In such embodiments, the retention member may present a convex surface to the tissue surrounding the opening to the anatomical passage to anchor the implantable medical device in place at the deployment site. Even more particularly, in accordance with various principles of the present disclosure, a retention member of an implantable medical device may have a bowl-shape, “mushroom,” or “umbrella” configuration, presenting an extended convex tissue-contacting surface for engaging and anchoring with respect to tissue at the deployment site. The inner surface of the retention member wall may be concave. The configuration of a retention member with at least a convex outer, tissue-contacting surface, and optionally also a concave inner surface (radially-inwardly directed, on the opposite side of the retention member wall), may result in a shape/configuration resistant to inversion. In such a bowl-shaped configuration, the retention member may be more resistant to migration from the deployment site than in an inverted configuration. As may be appreciated, the radially-outward extent of the tissue-contacting surface from the midsection and/or longitudinal axis of the implantable medical device to a free end or edge of the retention member (e.g., a terminal end or edge of the surface or structure in general) is sufficiently long, and provides sufficient surface area for engagement with tissue at the deployment site to retain the implantable medical device in place and to resist migration from the deployment site. For purposes of the present disclosure, terms such as anchor, affix, associate, deploy on, engage hold, retain, secure, etc. (and other grammatical forms thereof), may be used interchangeably herein without intent to limit.

Moreover, it will be appreciated that the extension of the retention member radially from the longitudinal axis of the implantable medical device may also contribute to the ability of the retention member to retain the implantable medical device in place with respect to a deployment site such as an anatomical passage. In order for the retention member to ride with anatomical forces in accordance with various principles of the present disclosure, while also retaining the implantable medical device in place at the deployment site, the retention member should have a radial extent (transverse to the longitudinal axis of the midsection and/or the device as a whole) sufficiently large to retain the implantable medical device in place at the deployment site. For instance, in some aspects, a retention member formed in accordance with various principles of the present disclosure to flex in response to anatomical forces also has an outer dimension when flexed, and/or may even have a portion which flexes outwardly, such that the implantable medical device presents a cross-sectional dimension or area sufficient to retain the implantable medical device in place with respect to the deployment site.

In view of the above, an implantable medical device formed in accordance with various principles of the present disclosure is sized, shaped, configured, and/or dimensioned to withstand forces applied thereto, such as to flex, absorb, ride with, etc., such forces, without impacting the ability of the retention member to retain the implantable medical device in place with respect to the deployment site thereof. Such dimensions may be determined by one of ordinary skill in the art, generally depending on the nature and location of the deployment site and anatomical structures thereof, without undue experimentation.

In some embodiments, the retention member of an implantable medical device is configured to ride with anatomical forces applied thereto to be compressed by such forces, yet to return to an initial configuration upon such force subsiding. For instance, the retention member may be shiftable from an expanded configuration to a compressed configuration upon application of radially-inwardly directed forces thereto, yet may automatically return to the expanded configuration upon release or reduction of the force applied thereto. In some embodiments, the retention member is formed from a shape memory material returning the retention member to a neutral configuration, such as an expanded configuration (relative to the compressed configuration the retention member undergoes in response to anatomical forces applied thereto), resistant to migration.

In some embodiments, a retention member additionally or alternatively is movable with respect to the midsection of the implantable medical device. For instance, the retention member may be rotatable with respect to the midsection, and/or pivotable in and out of a plane transverse to the midsection. In some embodiments, the retention member is formed separately from the body section of the implantable medical device and coupled thereto, either directly or in directly, in a manner facilitating various degrees of freedom of movement of the retention member to ride with anatomical forces impacting the retention member. In some embodiments, the midsection is elastic to allow the retention member to be pulled generally longitudinally with respect to the midsection. The midsection may be formed to limit, occlude, exclude, etc., flow of materials therethrough, depending on the nature of the treatment for which the implantable medical device is deployed.

Various embodiments of implantable medical devices, systems, and methods will now be described with reference to examples illustrated in the accompanying drawings. Reference in this specification to “one embodiment,” “an embodiment,” “some embodiments,” “other embodiments,” etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments,” “other embodiments,” etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

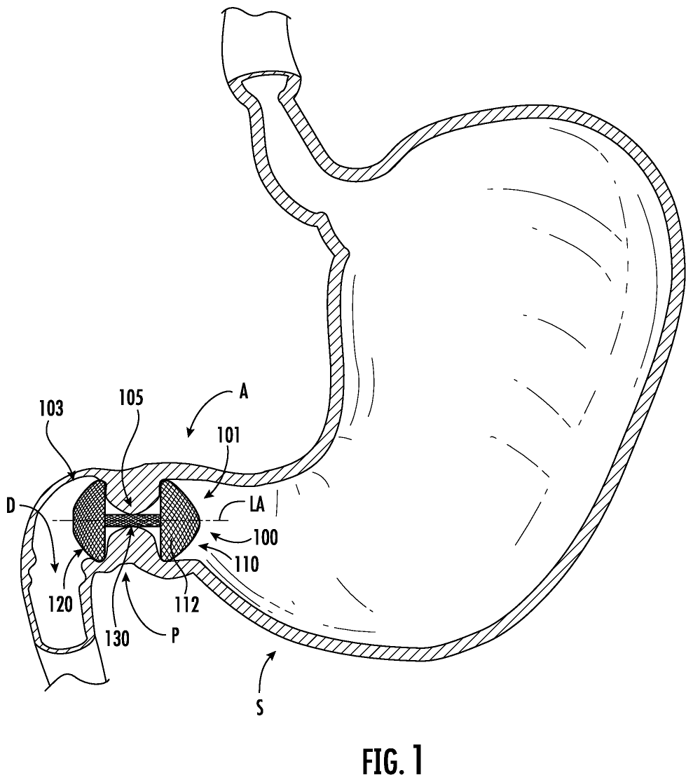

Turning now to the drawings, an example of an embodiment of an implantable medical device 100 is illustrated in FIG. 1 in an example of an embodiment of a gastrointestinal system, though other deployment sites are within the scope and content of the present disclosure. More particularly, the illustrated example of an embodiment of an implantable medical device 100 includes a first end 101 positioned within a stomach S, a second end 103 positioned within a duodenum D, and a midsection 105 positioned within a pylorus P. A first retention member 110 extends from the first end 101 of the implantable medical device 100 radially outwardly with respect to the longitudinal axis LA of the implantable medical device 100 and/or midsection 105 (referenced herein simply as the longitudinal axis LA for the sake of convenience without intent to limit, unless explicitly stated). In the illustrated example of an embodiment, the first retention member 110 is configured to engage with a tissue wall of the stomach S (e.g., the antrum thereof) surrounding the entry into the pylorus P. A second retention member 120 extends from the second end 103 of the implantable medical device 100 radially outwardly with respect to the longitudinal axis LA and is configured to engage with tissue wall of the duodenum D. Although the second retention member 120 is illustrated as generally similar to the first retention member 110, it will be appreciated that the second retention member 120 may have a different configuration than the first retention member 110, as will be discussed in further detail below. The extent of the illustrated implantable medical device 100 along the midsection 105 and between the retention members 110, 120 is referenced herein as the body 130 of the implantable medical device 100 without intent to limit. It will be appreciated that a “body” of an implantable medical device 100 formed in accordance with various principles of the present disclosure may be considered to include or not include the retention members 110, 120, the present disclosure not being restricted in this aspect.

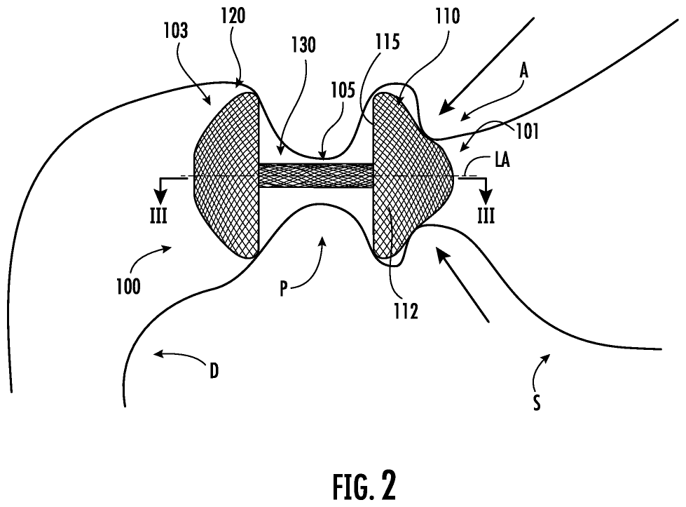

An implantable medical device 100 of the present disclosure, and particularly one or both of the retention members 110 thereof, may be subjected to regular forces, such as anatomical forces (internally and/or externally generated). For example, the illustrated implantable medical device 100 may be subjected to radially-inwardly-directed biological/anatomical forces. More particularly, when deployed in the environment illustrated in FIG. 1, at least the first retention member 110 is regularly subjected to peristaltic waves from the stomach S, particularly, the antrum A. In accordance with various principles of the present disclosure, at least the first retention member 110 (within the anatomical site exhibiting regular and/or strong forces on the implantable medical device 100, such as the antrum A) presents a convex tissue-contacting surface 112 for engaging and anchoring the implantable medical device 100 with respect to the deployment site, as illustrated in FIG. 1 and FIG. 2. More particularly, in contrast with prior implantable medical devices deployed in generally dynamic anatomical sites such as is the pylorus P, the tissue-contacting surface 112 does not present sharp or angular edges (e.g., generally clearly-defined edges, such as formed by surfaces joined at less than about 145° with respect to each other) for contact with tissue which may exert localized forces on such tissue-contacting surface 112. Instead, the tissue-contacting surface 112 presents a generally convex surface extending radially-outwardly from the longitudinal axis LA. Moreover, the first retention member 110 is sufficiently compliant and the tissue-contacting surface 112 thereof extends a sufficient distance radially-outwardly from the longitudinal axis LA and along the longitudinal axis LA and towards the midsection 105 to present a convex surface area with a sufficiently large radius of curvature to flex and dissipate forces applied thereto. As may be appreciated, the size, shape, configuration, and/or dimensions of the retention member 110 and the tissue-contacting surface 112 thereof, and/or stiffness and/or flexibility of the retention member 110, may be selected such that the tissue-contacting surface 112 is contacted over a sufficient area to dissipate forces impacting the retention member 110. Such size, shape, configuration, and/or dimensions, and/or stiffness/flexibility may be determined by one of ordinary skill in the art such as with reference to the nature of the deployment site without undue experimentation. It will be appreciated that references herein to various features and characteristics of the first retention member of an implantable medical device formed in accordance with various principles of the present disclosure may be applied to the second retention member as well.

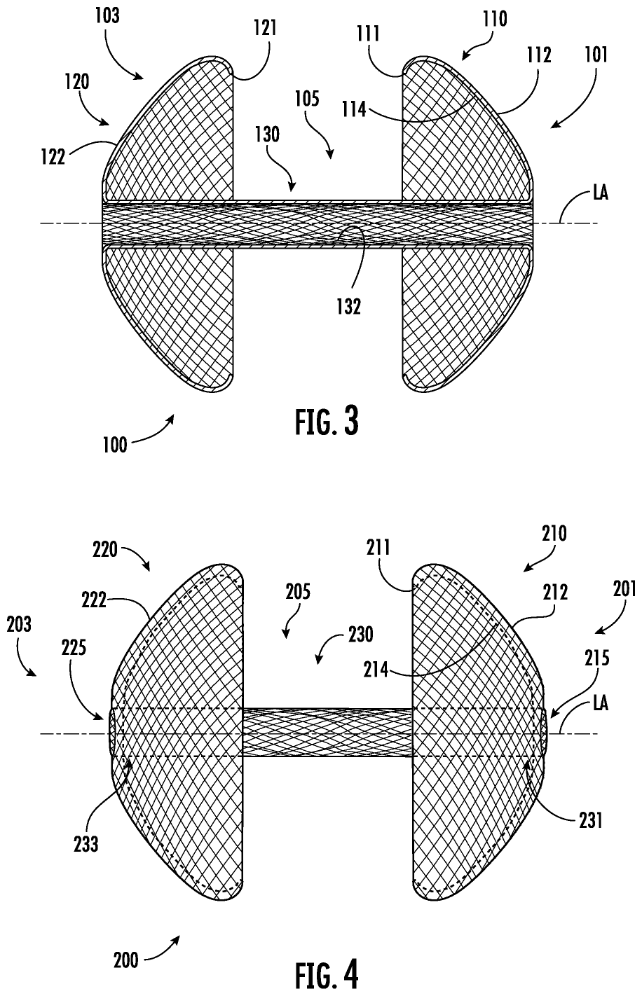

In accordance with further aspects of the present disclosure, the generally convex tissue-contacting surface 112 of a retention member 110 formed in accordance with various principles of the present disclosure is configured to resist inversion of the retention member 110 and thereby to resist migration which may occur if the retention member 110 is inverted with a typically thereby narrower dimension in a direction transverse to the longitudinal axis LA. For instance, the retention member 110 may be configured such as to form a generally umbrella-type configuration. For instance, the outer tissue-contacting surface 112 of the retention member 100 is convex, whereas the interior surface may be generally concave, such as illustrated in FIG. 3 and FIG. 6, and described in further detail below. As with umbrellas, such shape generally resists inversion. As may be appreciated by those of ordinary skill in the art, dimensions, proportions, etc., tensile strength, material composition, cross sectional shape as well as bending stiffness and/or flexibility of the retention member 100 may be selected, such as with reference to the ultimate deployment site thereof, to assure the desired resistance to inversion.

An example of an embodiment of a first retention member 110 formed in accordance with various principles of the present disclosure is illustrated in FIG. 2. As may be appreciated, the generally convex tissue-contacting surface 112 has sufficient area to withstand forces (indicated by the generally inwardly directed arrows) applied thereto from the deployment site (in this case the antrum A of the stomach S). Moreover, the first retention member 110 and tissue-contacting surface 112 are sufficiently flexible/compliant to generally conform with the shape of the tissue wall applying the force and/or to otherwise dissipate (“ride with”) the forces applied thereto rather than to resist such forces. As such, forces applied to the implantable medical device 100 are dissipated, and forces and stresses do not concentrate at particular regions of the first retention member 110 as may occur with prior retention members of similar implantable medical devices. Moreover, if the first retention member 110 is sufficiently compliant, then the tissue-contacting surface 112 may withstand forces applied thereto without propagating such forces to the free end 115 of the first retention member 110. It will be appreciated that reference to the free end 115 is intended to encompass reference to the peripheral free edge of the first retention member 110.

In some embodiments, an implantable medical device 100 is formed from a plurality of strands or wires or filaments or ribbons (such terms being used interchangeably herein without intent to limit) which are braided or woven or twisted or wrapped or intertwined or knitted or looped (e.g., bobbinet-style) or knotted or otherwise to form the wall of the implantable medical device 100. The free ends of the filaments forming the implantable medical device 100 may be welded to retain the filaments in place with respect to one another. For the sake of convenience, and without intent to limit, reference is made generally to formation of an implantable medical device with interwoven filaments to refer to the general manner of forming an implantable medical device from multiple interconnected elements. In contrast, an implantable medical device 100 may alternatively be formed from a single, monolithic element such as a laser cut tube, cut or otherwise shaped into the desired configuration. The filaments may be formed of generally biocompatible materials such as a variety of appropriate metals (e.g., nickel-titanium alloy, such as Nitinol, or another generally shapable metal, such as stainless steel) or polymers or combinations thereof known to those of ordinary skill in the art. In some embodiments, the material of the implantable medical device is a shape memory and/or heat formable material allowing the retention member a degree of resiliency so that the retention member does not plastically deform and returns to an expanded configuration to resist migration from the deployment site and thereby to retain the implantable medical device in place.

A reduction of forces propagated to the free end 115 of the first retention member 110 generally reduces forces which may affect the integrity of the welds of filaments forming a woven implantable medical device 100 and thereby affecting the integrity of the implantable medical device 100. As such, a configuration of a compliant first retention member 110 and/or a first retention member 110 with a convex tissue-contacting surface 112 in accordance with various principles of the present disclosure reduce stresses which may otherwise affect the structural integrity of the wall thereof.

As may be appreciated with reference to the cross-sectional view in FIG. 3, along line III-III of the example of an embodiment of an implantable medical device 100 illustrated in FIG. 2, at least the first retention member 110 may also have a generally concave inner surface 114 generally facing in a direction opposite the direction in which the tissue-contacting surface 112 faces. The combination of a generally convex tissue-contacting surface 112 and a generally concave inner surface 114 permits the first retention member 110 to resist inversion, similar to a manner in which an umbrella may resist inversion. Such configuration may counterbalance/offset various more compliant features of the first retention member 110.

An implantable medical device 100 formed in accordance with various principles of the present disclosure may be formed from a generally tubular member defining the tubular body 130. To form one or both of the retention members 110, 120, an end region of the tubular member forming the tubular body 130 may be inverted and pulled in a direction towards the other end of the tubular member, into a first retention member 110 such as illustrated in FIG. 3. More particularly, the free end or circumferential edge of the tubular member may be inverted and pulled a sufficient distance toward a midsection thereof (between the ends of the tubular member) to form a retention member 110, 120 on either end of a body 130 which resists returning to the initial tubular configuration. It will be appreciated that the ends of the body 130 formed from the tubular member are not necessarily the ends of the tubular member from which the body 130 is formed, as the ends of the tubular member may be free ends and thus may be define free ends of the retention members 110, 120 formed by inverting the free ends of the tubular member. In some embodiments, the inverted ends of the tubular member are also shaped and formed to define a generally convex tissue-contacting surface 112, 212 such as illustrated in FIG. 3. The free end 111, 121 of the respective retention members 110, 120 is extended a sufficient distance towards the opposite retention member 120, 110 to provide sufficient area for the respective tissue-contacting surfaces 112, 122 to anchor the implantable medical device 100 as well as to provide sufficient structure (length, bending stiffness, etc.) to resist inversion or “unrolling” of the first retention member 110 to its starting position (which may be less resistant to migration of the implantable medical device 100). In some embodiments, the free ends 111, 121 may be directed inwardly towards the longitudinal axis LA. In embodiments in which the implantable medical device 100 is formed from weaving or otherwise interengaging filaments, the filaments may be woven to form a generally tubular member from which the implantable medical device 100 may be formed. The end of the tubular member may then be inverted, and optionally also stretched, into the inverted umbrella-like configuration illustrated in FIG. 3.

It will be appreciated that even if the body 130 of an implantable medical device 100 formed in accordance with various principles of the present disclosure is tubular and defines a lumen therethrough, such as illustrated in FIG. 2 and FIG. 3, the lumen need not be configured to allow passage of material therethrough readily if at all. In some embodiments the lumen may be occluded such as to inhibit or to prevent flow of materials therethrough. For instance, in the example of an embodiment illustrated in FIG. 2 and FIG. 3, the inner diameter of the lumen 132 through the body 130 of the implantable medical device 100 may be narrowed, constricted, or occluded to reduce or to prevent flow of materials therethrough. In the example of an embodiment illustrated in FIG. 1, occlusion of the implantable medical device 100 may be desirable to inhibit or to prevent flow of gastric materials from the stomach S into the duodenum D (such as in conjunction with bariatric treatments, including formation of a gastric bypass formed between the stomach S and the jejunum).

Various modifications to an implantable medical device with a compliant retention member capable of riding with anatomical forces exerted (generally radially-inwardly) thereon as well as resisting migration of the implantable medical device (generally longitudinally) are within the scope and spirit of the present disclosure. For instance, the retention members which anchor the implantable medical device in place with respect to the deployment site may be formed separately from the body portion which extends across a deployment site such as an anatomical passage. Additionally or alternatively, the retention members and the body may be formed in a different manner, such as from different materials with optionally different properties.

An example of another embodiment of an implantable medical device 200 formed in accordance with various principles of the present disclosure with at least one generally compliant retention member 210, 220 is illustrated in FIG. 4. The compliant retention members 210, 220 may have any of the properties of the retention members 110, 120 described above, in any combination thereof, which allow compliant riding with forces applied thereto while resisting migration and retaining the implantable medical device in place at its deployment site. Accordingly, for the sake of brevity, reference is made to the above descriptions of properties of a compliant retention member formed in accordance with various principles of the present disclosure as applicable to retention members 210, 220 of FIG. 4. In the illustrated example of an embodiment, the tissue-contacting surfaces 212, 222 of the retention members 210, 220 extend from a midregion 215, 225 positioned generally along the longitudinal axis LA, radially outwardly from the longitudinal axis LA, and generally longitudinally toward the midsection 235 of the body 230 and the opposite retention member 220, 210. The retention members 210, 220 may be generally bowl-shaped to present a generally convex tissue-contacting surfaces 212, 222 to tissue at the deployment site to absorb forces applied thereto.

In the example of an embodiment of an implantable medical device 200 illustrated in FIG. 4, one or both of the retention members 210, 220 are formed separately from the body 230 of the implantable medical device 200 extending therebetween. As such, at least one of the retention members 210, 220 is more readily movable with respect to the body 230 than in prior implantable medical devices formed for similar purposes. For instance, at least one of the retention members 210, 220 may be rotatable and/or pivotable (e.g., in and out of a plane transverse to the longitudinal axis LA) with respect to the body 230. The ends 231, 233 of the body 230 may be coupled to the retention members 210, 220 in any of a variety of manners allowing relative movement between the body 230 and the retention members 210, 220. The ends of the body 230 may be bonded, looped, interwoven, hooked, or otherwise engaged with the retention members 210, 220, either directly or with another element (e.g., a suture), to hold the components of the implantable medical device 200 together, such as depending on the manner in which the retention members 210, 220 and body 230 are formed (as described in further detail below). In some embodiments, the ends 231, 233 of the body 230 are coupled with generally central midregions 215, 225 of the retention members 210, 220. In some embodiments, the body 230 is insertable into a generally central region of the retention members 210, 220 to allow rotation of the retention members 210, 220 with respect to the body 230, yet to prevent separation of the retention members 210, 220 from the body 230. For instance, the free ends of the body 230 may be enlarged (e.g., after being inserted through the retention members 210, 220) to maintain a connection with the retention members 210, 220. It will be appreciated that the manner of connecting the body 230 with the retention members 210, 220 may be a generally flexible, generally not rigid, connection, thereby generally avoiding stress concentrations and allowing a degree of movement and “riding” with anatomical forces applied thereto.

Additionally or alternatively, different materials may be used to form the retention members 210, 220 and the body 230. In some embodiments, the body 230 of the implantable medical device 200 of FIG. 4 may be formed of an elastic material allowing stretching to accommodate anatomical forces impacting the implantable medical device 200 rather than resisting such forces. As such, not only are the retention members 210, 220 able to “ride” with anatomical forces rather than resist such forces, but the body 230 may also “ride” with anatomical forces impacting the implantable medical device 200.

Additionally or alternatively, the retention members 210, 220 and the body 230 of the example of an embodiment of an implantable medical device 200 illustrated in FIG. 4 may be formed in different manners. For instance, the retention members 210, 220 may be formed from woven filaments, similar to the retention members 110, 120 of the implantable medical device 100 illustrated in FIG. 2 and FIG. 3. In contrast, instead of being formed from woven filaments, the body 230 of the implantable medical device 200 illustrated in FIG. 4 may be formed from an elastic rod or even an elastic band (e g, similar to a rubber band) or tether element or the like. The ends 231, 233 of the body 230 (e.g., ends of filaments forming the body 230 and/or ends of a band forming the body 230) may be looped about elements, such as filaments, forming the retention members 210, 220 to hold the body 230 and retention members 210, 220 together in a manner allowing relative movement therebetween.

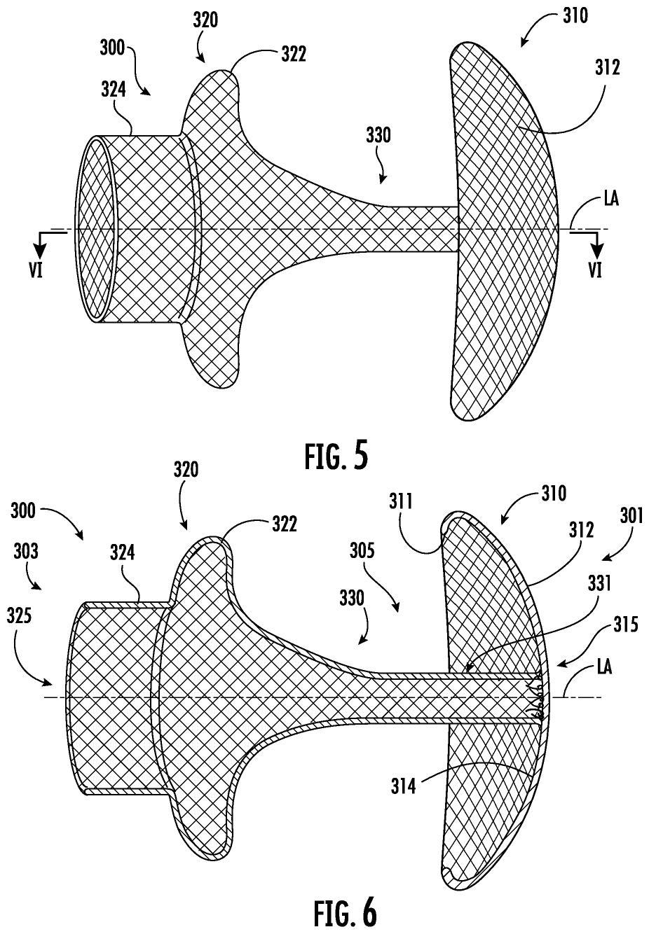

As noted above, the retention members of an implantable medical device formed in accordance with various principles of the present disclosure need not have the same configurations. For instance, the example of an embodiment of an implantable medical device 300 illustrated in FIG. 5 and FIG. 6 may have differently shaped retention members 310, 320. The first retention member 310 may be bowl-shaped and formed similar to the bowl-shaped retention members 110, 210 described above and illustrated in FIG. 2, FIG. 3, and FIG. 4. For instance, the first retention member 310 may extend from a generally central region 315 at the first end 301 of the body 330, radially outwardly from the longitudinal axis LA, and towards the midsection 305 of the body 330. Moreover, the first retention member 310 may be compliant and flexible to flex inwardly in response to radially inwardly forces exerted thereon and thereby to conform to the anatomical structure at which the implantable medical device 300 is deployed. The first retention member 310 preferably does not plastically deform, and is able to return to an expanded configuration upon reduction or withdrawal of the anatomical forces applied thereto. As such, the first retention member 310 is configured to “ride” radially-inwardly directed forces, such as peristaltic waves, impacting the implantable medical device 300. Like the retention members 110, 210 described above, the first retention member 310 is sized, shaped, configured, and/or dimensioned to resist migration of the implantable medical device 300 even when being compressed or otherwise deformed in response to forces applied thereto. It will be appreciated that such properties of a retention member are applicable to the above-described retention members, and the above-described properties of previously described examples of embodiments of retention members are applicable to the retention member 310 illustrated in FIG. 5 and FIG. 6.

In deployment sites in which the second retention member 320 is subjected to anatomical forces of a different nature than those impacting the first retention member 310, the second retention member 320 may have a different shape and configuration than that of the first retention member 310. For instance, the example of an embodiment of an implantable medical device 300 illustrated in FIG. 5 and FIG. 6 is configured to be implanted across a pylorus P with the second retention member 320 extending into a duodenum D (FIG. 1). The second retention member 320 includes a tissue-contacting surface 322 extending radially-outwardly from the longitudinal axis LA and configured to engage the duodenum D to hold the implantable medical device 300 in place with respect to the duodenum D and against migration. However, because the deployment site of the second retention member 320 is the duodenum D rather than the stomach S, the second retention member 320 may be more elongated (for example, along the longitudinal axis LA) than the first retention member 310, e.g., with an extended generally cylindrical tissue-contacting surface 324. Such elongated configuration of the second retention member 320 may distribute and dissipate peristaltic forces from the duodenum D along the length thereof better than the generally shorter retention member 310 configured for placement on the gastric side of the pylorus P. In some embodiments, the diameter of the tissue-contacting surface 322 of the second retention member 320 is closer to the diameter of the generally cylindrical tissue-contacting surface 324 than as illustrated in FIG. 5 and FIG. 6, and, in some embodiments, these diameters may even be substantially the same. Additionally or alternatively, the transition from the body 330 to the second retention member 320 may be gradual, as illustrated in FIG. 5 and FIG. 6, or more angular (e.g., similar to the transition to the first retention member 310), depending on the anatomical structure in which the second retention member 320 is to be deployed. Moreover, the antrum A typically exerts stronger peristaltic forces than exerted by the duodenum D, thereby requiring modifications to the gastric retention member 310 (such as the features described herein) not necessarily required in the duodenal retention member 320.

The body 330 and retention members 310, 320 of the implantable medical device 300 illustrated in FIG. 5 and FIG. 6 may be formed as a single component or from separate components coupled together. For instance, the body 330 of the implantable medical device 300 may be formed from a tubular member, with the ends thereof expanded to form the retention members 310, 320 (such as described with respect to the implantable medical device 100 illustrated in FIG. 2 and FIG. 3). More particularly, the body 330 may be formed from a generally tubular member (e.g., from interwoven filaments) with an end along the first end 301 of the implantable medical device 300 inverted to form the first retention member 310 with the free end 315 thereof spaced inwardly from the first end 301 of the implantable medical device 300 and towards the midsection 305 of the implantable medical device 300. The body 330 may be expanded along the second end 303 of the implantable medical device 300 to form the second retention member 320. Alternatively, the body 330 and the second retention member 310 of the implantable medical device 300 illustrated in FIG. 5 may be formed together, but separately from the first retention member 310. For instance, the body 330 and the second retention member 310 may be formed from a tubular member expanded at one end to form the second retention member 320. The first retention member 310 may be separately formed in a bowl-shaped configuration with a convex tissue-contacting surface 312, and then coupled with the body 330 and the second retention member 310. The first retention member 310 may be formed from interwoven filaments or in any other manner allowing compliant and resilient compression in response to radially-inwardly directed forces, and the ability to return to an expanded configuration upon forces applied thereto subsiding, and to resist migration of the implantable medical device 300 from its deployment site, such as in manners described above.

The formation of the first retention member 310 of the implantable medical device 300 illustrated in FIG. 5 and FIG. 6 separate from the body 330 may afford greater degrees of freedom of movement to the first retention member 310 than may be afforded if formed integrally with and as part of the body 330. Such freedom of movement may allow the first retention member 310 to ride with anatomical forces impacting the implantable medical device 300 with less stress on the first retention member 310 than withstood by prior art devices used for similar purposes. The body 330 and the first retention member 310 may be connected in a variety of manners affording multiple degrees of freedom of movement. For instance, if the body 330 and the first retention member 310 are formed from interwoven filaments, the free ends of filaments forming the body 330 may be looped around or otherwise interwoven or interegaged with the first retention member 310 (e.g., with filaments forming the first retention member 310) to couple the body 330 and the first retention member 310 together. For instance, as illustrated in further detail in FIG. 6, ends of filaments at the first end 311 of the body 330 may be looped around filaments forming the first retention member 310 to couple the body 330 to the first retention member 310. Such connection allows rotation and/or pivoting of the first retention member 310 with respect to the body 330, such as in manners described herein with respect to various retention members formed in accordance with various principles of the present disclosure (such as the implantable medical device 200 illustrated in FIG. 4). In some embodiments, the first retention member 310 may be coated with a biocompatible material, such as silicone, which may bond the free ends of the filaments of the body 330 with respect to the first retention member 310 and/or inhibit tissue ingrowth with respect to the first retention member 310 (thereby facilitating movement with respect to tissue at the deployment site), and/or inhibit or prevent flow of materials therethrough. Alternatively, if the first retention member 310 is not formed with interwoven filaments, then the body 330 may be coupled thereto in other manners known to those of ordinary skill in the art, such as described above with reference to the example of an embodiment illustrated in FIG. 4.

As noted above, it will be appreciated that various features of the above-described examples of embodiments can be interchanged with those of others of the above-described examples of embodiments, such features being arranged and operating in substantially the same or similar manners. Accordingly, common elements with common functions have been indicated with the same reference characters differing in value by 100, with descriptions of features of one embodiment being generally applicable to similar features of other embodiments, such descriptions not being repeated for the sake of brevity and convenience, and without intent to limit.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. It will be appreciated that reference to a body passage includes naturally-existing passages (e.g., the colon) as well as medically-created passages (e.g., a passage created with the use of a medical instrument, and not existing without medical intervention) or otherwise.

It will be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Various further benefits of the various aspects, features, components, and structures of an implantable medical device such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more”, and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable medical device comprising:
a body having a first end, a second end, and a midsection therebetween;
a first retention member extending from the first end of said body; and
a second retention member extending from the second end of said body;
wherein:
said first retention member extends radially outwardly from the first end of said body and toward the midsection of said body to define a tissue-contacting surface with a surface area and flexibility sufficient to absorb radially-inwardly directed anatomical forces applied thereto; and
said first retention member is configured to resist being inverted into a configuration with the free end thereof extending away from the first end and the midsection of said body.

2. The implantable medical device of claim 1, wherein said first retention member is formed of a compliant material capable of flexing with radially-inwardly directed anatomical forces applied thereto.

3. The implantable medical device of claim 2, wherein said first retention member is compressible from an initial expanded configuration in response to application of radially-inwardly forces applied thereto, and is formed of a resilient material returning said first retention member to the expanded configuration upon the force subsiding.

4. The implantable medical device of claim 3, wherein said first retention member presents a convex tissue-contacting surface to tissue at the deployment site, and is configured and dimensioned to resist migration from the deployment site.

5. The implantable medical device of claim 1, wherein said body is formed of a tubular member, and at least said first retention member is formed by inverting an end of the tubular member and extending the free end of the inverted end toward the midsection of the tubular member a sufficient distance to cause the inverted end of the tubular member to resist returning to its initial uninverted tubular configuration.

6. The implantable medical device of claim 5, wherein said tubular member is formed from a plurality of interwoven filaments.

7. The implantable medical device of claim 1, wherein said first retention member is formed from a bowl-shaped element separate from said body.

8. The implantable medical device of claim 7, wherein said first retention member is movable with respect to said body.

9. The implantable medical device of claim 7, wherein said body is formed from an elastic material allowing said first retention member to be moved away from said second retention member.

10. The implantable medical device of claim 1, wherein the tissue-contacting surface of said first retention member is convex.

11. An implantable medical device configured to be implanted with respect to a pylorus of a patient, the implantable medical device comprising:
a body having a first end, a second end, and a midsection extending therebetween;
a gastric retention member extending radially-outwardly from the first end of said body; and
a duodenal retention member extending radially-outwardly from the second end of said body;
wherein:
said gastric retention member has a tissue-contacting surface extending radially outwardly from the first end of said body and toward the midsection of said body to a free edge of said gastric retention member spaced from the first end of said body in a direction toward the midsection of said body a sufficient extent to remain in such configuration when said implantable medical device is implanted with respect to the patient's pylorus with said gastric retention member positioned within the patient's stomach; and
said gastric retention member is resiliently compliant to contract from an expanded configuration to a compressed configuration upon application of peristaltic forces thereto from the antrum of the patient's stomach, and to return to the expanded configuration upon passing of a peristaltic wave from said gastric retention member.

12. The implantable medical device of claim 11, wherein said gastric retention member presents a convex tissue-contacting surface to the antrum of the patient's stomach.

13. The implantable medical device of claim 11, wherein:
said implantable medical device is formed from a tubular member having a first end, a second end, and a midsection therebetween; and
said gastric retention member is formed by inverting the first end of the tubular member and pulling the free end thereof towards the second end of the tubular member a sufficient distance to remain in the inverted configuration when implanted with respect to the antrum of the patient's stomach and when compressed by a peristaltic wave of the antrum.

14. The implantable medical device of claim 11, wherein said body and at least said gastric retention member are formed separately and coupled together to allow relative movement therebetween.

15. The implantable medical device of claim 14, wherein said body is formed of an elastic material allowing said gastric retention member to move away from said duodenal retention member.

16. A method of at least partially occluding flow of materials through a pylorus of a patient, said method comprising:
deploying a body of an implantable medical device across the pylorus;
deploying a gastric retention member of the implantable medical device in the antrum of the patient, the gastric retention member having a tissue-contacting surface extending radially-outwardly from a first end of the body of the implantable medical device and towards a midsection of the body, the gastric retention member being formed of a compliant material compressing in response to peristaltic contractions of the antrum and returning to an expanded configuration upon passing of a peristaltic contraction of the antrum; and
deploying a duodenal retention member of the implantable medical device in the duodenum of the patient, the duodenal retention member extending radially-outwardly from a second end of the body of the implantable medical device.

17. The method of claim 16, wherein the implantable medical device is formed from a tubular member having a first end and a second end, with the first end inverted to form the gastric retention member of the implantable medical device, said method comprising placing the inverted first end in contact with the antrum with the free end of the tubular member positioned inwardly of the first end of the tubular member in a direction toward the second end of the tubular member.

18. The method of claim 16, wherein the gastric retention member presents a convexly curved tissue-contacting surface, said method comprising placing the concavely-curved tissue-contacting surface in contact with the patient's antrum for contracting in response to peristaltic waves applied thereto.

19. The method of claim 18, wherein the gastric retention member extends a sufficient distance from the first end of the body to the midsection of the body to resist inversion causing the gastric retention member to extend away from the midsection of the body.

20. The method of claim 16, wherein the implantable medical device is formed from a body separate from the gastric retention member such that upon placement of the implantable medical device with respect to the patient's pylorus, the gastric retention member is capable of movement with respect to the body of the implantable medical device.
